(19) United States
(12) Patent Application Publication
(10) Pub. No.: US 2018/0185332 A1
(43) Pub. Date: Jul. 5, 2018

(54) Leukotriene Receptor Antagonists and Their Derivatives for Use as Antibacterial Agents

(71) Applicant: University of East London, London (GB)

(72) Inventors: John Terry George, London (GB); Akintunde Emiola, London (GB)

(21) Appl. No.: 15/100,073

(22) PCT Filed: Nov. 28, 2014
(86) PCT No.: PCT/GB2014/053541

§ 371 Date: May 27, 2016

(30) Foreign Application Priority Data
Nov. 29, 2013 (GB) 1321089.3

(51) Int. Cl. A61K 31/405; A61K 31/41; A61K 31/381; A61P 31/04; A61P 29/00; A61P 11/06

(52) U.S. Cl. CPC A61K 31/405; A61K 31/41; A61P 11/06; A61P 31/04; A61P 29/00; A61K 31/381

(57) ABSTRACT

The present invention relates to leukotriene receptor antagonists, in particular zafirlukast and its derivatives, for use in the treatment of Gram-positive bacterial infections, wherein the bacterial infections are not a Mycobacterium or Corynebacterium infections, as well as pharmaceutical compositions comprising the same and corresponding methods of treatment.

The present invention relates to leukotriene receptor antagonists, in particular zafirlukast and its derivatives, for use in the treatment of bacterial infections and as anti-bacterial and antibiotic agents.

Antibiotic resistance is a major problem in the global fight against infectious diseases. The continual adaption of bacterial species has resulted in an increase in antibiotic-resistant strains, including strains of bacteria that are considered to be multi-drug resistant (MDR).

The emergence of antibiotic resistant strains has been worsened by such behaviours as inappropriate prescribing of antibiotics, poor patient compliance (the patient stops taking the antibiotic when the symptoms subside and do not finish the course prescribed by their doctor) or the lack of control in the supply of antibiotics to the public, for example, in those countries where antibiotics are available over the counter without prescription. Antibiotic use in veterinary medicine may also play a role.

Common strains of antibiotic-resistant bacteria include methicillin-resistant Staphylococcus aureus (MRSA), Streptococcus pneumoniae, (resistant to penicillin and other beta-lactams) and multi-drug resistant Enterococcus faecalis and Clostridium difficile, to name but a few.

Leukotrienes are biologically active compounds derived from arachidonic acid. They were originally discovered in leukocytes, although they are also found in other immune cells. They are a family of eicosanoid inflammatory mediators. They are known to contribute to the pathophysiology of asthma and may cause or potentiate symptoms of increased mucus secretion, bronchoconstriction, air flow obstruction and infiltration of inflammatory cells into the airway wall. Leukotriene antagonists inhibit leukotriene receptors and are known for use in the treatment of asthma or bronchitis, although are less effective than corticosteroids.

Zafirlukast is a leukotriene antagonist used for the treatment of asthma, often in conjunction with an inhaled steroid and/or long-acting bronchodilator. It is marketed by AstraZeneca under the names Accolate, Accoleate and Vanticon. It is administered orally and has a plasma half-life of approximately 10 hours. Zafirlukast blocks the action of cysteinyl leukotrienes on CysLT1 receptors.

Pinault et al noted that zafirlukast inhibits the complexation of Lsr2 (a regulatory protein involved in multiple cellular processes, including cell wall biosynthesis) with DNA and growth of Mycobacterium tuberculosis (Pinault et al, Antimicrobial Agents and Chemotherapy, 2013, 57(5):2134-2140). In particular, the authors noted that zafirlukast inhibits the growth of Mycobacterium smegmatis and Mycobacterium tuberculosis, although it had no effect on the growth of E. coli. The authors also found that zafirlukast exhibits bactericidal activity against M. smegmatis but these effects were attributed to the inhibitory effect of zafirlukast on Lsr2.

WO 2013/055674 discloses methods of treating bacterial infections using zafirlukast, in particular Mycobacterium infections and Corynebacterium infections.

There is a need in the art for the provision of new antibiotics or the discovery of novel uses of known compounds as antibiotics to address the growing and serious problem of bacterial resistance to current treatment regimens. The ability to treat infections of antibiotic-resistant bacteria would be particularly advantageous. Lower dosages are also desirable.

The present inventors have surprisingly found that leukotriene antagonists, such as zafirlukast and derivatives thereof, have antimicrobial activity against a range of bacteria including a number of opportunistic Gram-positive organisms, such as Staphylococcus aureus, methicillin-resistant Staphylococcus aureus, Enterococcus faecalis, Bacillus subtilis, Streptococcus pneumoniae, and Clostridium difficile.

Accordingly, in a first aspect of the invention there is provided a leukotriene antagonist for use in the treatment of bacterial infection. In particular, the leukotriene antagonist is for use as an antibiotic in the treatment of a Gram-positive bacterial infection. There is also provided a compound according to any one of Formulae I to III for use in the treatment of bacterial infection. Leukotriene antagonists and compounds according to any one of Formulae I to III are collectively referred herein to as “compounds used in the invention”. Leukotriene antagonists and compounds according to any one of Formulae I to III are used in the present invention as antibiotics, in particular bactericidal antibiotics. The antibiotic effect is achieved via direct action of the compounds on the bacteria infecting a patient. The compounds used in the invention are therefore surprisingly effective in targeting and treating the underlying infection and not just the symptoms of or immune response to the infection.

The compounds used in the present invention are particularly useful in the treatment of infections by Firmicutes, in particular Gram-positive Firmicutes. In some embodiments, the compounds are used to treat Staphylococcus spp., Enterococcus spp., Bacillus spp., Streptococcus spp. and Clostridium spp. infections. In some embodiments, the bacterial infections treated are infections by opportunistic bacteria, in particular, opportunistic Gram-positive bacteria. Nosocomial infections, including nosocomial Gram-positive infections, may be targeted in the present invention. The bacteria may be antibiotic-resistant or even multidrug-resistant strains. Specific infections that can be treated include Staphylococcus aureus, methicillin resistant Staphylococcus aureus, Enterococcus faecalis, Bacillus subtilis, Streptococcus pneumoniae and Clostridium difficile infections.

In some embodiments, the bacterial infections treated are infections of bacterial species that do not encode Lsr2, or an Lsr2-homologue. Bacterial species targeted in such embodiments therefore do not comprise Lsr2 or an Lsr2-homologue. Lsr2-homologs are found in, for example, actinobacteria, such as Streptomyces, Nocardia and Rhodococcus. Lsr2 has the accession number CCE39020.1 (in the European Nucleotide Archive). The sequence for Lsr2 of M. tuberculosis is:

MAKKVTVTLVDDFDGSGAADETVEFGLDGVTYEIDLSTKNATKLRGDLK
QWVAAGRRVGGRRRGRSGSGRGRGAIDREQSAAIREWARRNGHNVSTRG
RIPADVIDAYHAAT

Lsr2 is an H—NS protein that has a role in gene expression. The protein is functionally related to the H—NS superfamily, which is present in other bacterial species.

In some embodiments, the bacterial infections to be treated are not Mycobacterium spp. or Corynebacterium spp. infections. In some embodiments of the invention, the bacteria targeted are not actinobacteria. In some embodiments of the invention, the bacterial infections to be treated are not Streptomyces spp., Nocardia spp. or Rhodococcus spp. infections.

The bacterial infections may be chronic or acute bacterial infections, although the invention is particularly useful for the treatment of acute bacterial infections.

The present inventors have surprisingly found that the antimicrobial activity of leukotriene antagonists and the compounds of Formulae I to III are not dependent on the presence of Lsr2 or an Lsr2 homologue in the bacterial species being targeted. The present inventors have also found that such compounds are bactericidal and are not simply bacteriostatic. Suitably, the compounds of Formula I to III are leukotriene receptor antagonists.

Compounds of Formula I have the following structure:

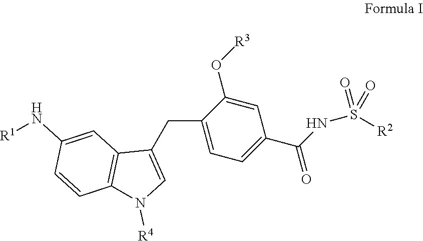

wherein:
R¹ is H, an optionally substituted C₁₋₄ alkyl, C(O)R′ or C(O)OR″;
   R′ is H or an optionally substituted C₁₋₄ alkyl or —NR^{III}R^{IV};
   R^{III} and R^{IV} are independently H, an optionally substituted C₁₋₄ alkyl, or are taken together to form a heterocyclic ring;
   R″ is H, an optionally substituted C₁₋₆ alkyl or an optionally substituted C₃₋₆ cycloalkyl or heterocycloalkyl;
R² is H, or an optionally substituted alkyl, cycloalkyl or heterocycloalkyl, aryl or heteroaryl;
R³ is H, or an optionally substituted C₁₋₄ alkyl; and
R⁴ is H, or an optionally substituted C₁₋₄ alkyl,
or pharmaceutically acceptable salts, esters, amides or prodrugs thereof.

R¹ is H, an optionally substituted C₁₋₄ alkyl, C(O)R′ or C(O)OR″, wherein R′ is H or an optionally substituted C₁₋₄ alkyl or NR^{III}R^{IV}, wherein R^{III} and R^{IV} are independently H, an optionally substituted C₁₋₄ alkyl, or are taken together to form a heterocyclic ring; and R″ is H, an optionally substituted C₁₋₆ alkyl or an optionally substituted C₃₋₆ cycloalkyl or heterocycloalkyl. Preferably, R¹ is selected from the group consisting of H, C(O)Me and C(O)OR″, wherein R″ is H or a cycloalkyl (preferably C₅) optionally substituted with OH.

R² is H, or an optionally substituted alkyl, cycloalkyl, heterocycloalkyl, aryl or heteroaryl. Preferably, R² is selected from the group consisting of aryl or heteroaryl optionally substituted with alkyl, halogen or amino. More preferably, R² is an aryl optionally substituted with CH₃.

In some embodiments, R¹ is selected from the group consisting of H, C(O)Me and C(O)OR″, wherein R″ is H or a cycloalkyl (preferably C₅) optionally substituted with OH and R² is an aryl optionally substituted with CH₃.

R³ is H, or an optionally substituted C₁₋₄ alkyl. Preferably, R³ is selected from the group consisting of H and Me.

R⁴ is H, or an optionally substituted C₁₋₄ alkyl. Preferably, R⁴ is a C₁₋₄ alkyl optionally substituted with —OH. More preferably, R⁴ is selected from the group consisting of H, Me and CH₂OH.

In some embodiments, R¹ is selected from the group consisting of H, C(O)Me and C(O)OR″, wherein R″ is H or a cycloalkyl (preferably C₅) optionally substituted with OH, R² is an aryl optionally substituted with CH₃, R³ is selected from the group consisting of H and Me and R⁴ is selected from the group consisting of H, Me and CH₂OH.

Compounds of Formula II have the following structure:

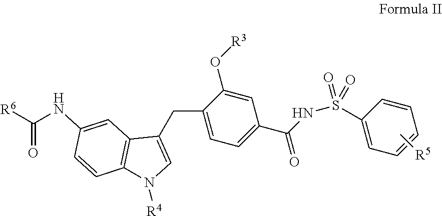

wherein:
R³ is H, or an optionally substituted C₁₋₄ alkyl;
R⁴ is H, or an optionally substituted C₁₋₄ alkyl;
R⁵ is H, or an optionally substituted C₁₋₄ alkyl; and
R⁶ is H, a C₁₋₄ alkyl, a heterocycloalkyl or OR′″, wherein R″ is H, C₁₋₆ alkyl or an optionally substituted C₃₋₆ cycloalkyl,
or pharmaceutically acceptable salts, esters, amides or prodrugs thereof.

R³ is H, or an optionally substituted C₁₋₄ alkyl. Preferably, R³ is selected from the group consisting of H and Me.

R⁴ is H, or an optionally substituted C₁₋₄ alkyl. Preferably, R⁴ is a C₁₋₄ alkyl optionally substituted with —OH. More preferably, R⁴ is selected from the group consisting of H, Me and CH₂OH.

R⁵ is H, or an optionally substituted C₁₋₄ alkyl. Preferably, R⁵ is H or Me.

R⁶ is H, a C₁₋₄ alkyl, a heterocycloalkyl or OR′″, wherein R′″ is H, C₁₋₆ alkyl or an optionally substituted C₃₋₆ cycloalkyl. Preferably, R⁶ is OR′″, wherein R″ is a C₃₋₆ cycloalkyl optionally substituted with OH.

In some embodiments, R³ is selected from the group consisting of H and Me; R⁴ is selected from the group consisting of H, Me and CH₂OH; R⁵ is H or Me and R⁶ is OR′″, wherein R′″ is a C₃₋₆ cycloalkyl optionally substituted with OH.

Compounds of Formula III have the following structure:

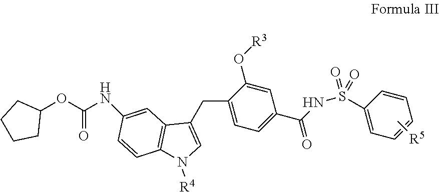

wherein:
R³ is H, or an optionally substituted C₁₋₄ alkyl;
R⁴ is H, or an optionally substituted C₁₋₄ alkyl; and
R⁵ is H, or an optionally substituted C₁₋₄ alkyl,
or pharmaceutically acceptable salts, esters, amides or prodrugs thereof.

R³ is H, or an optionally substituted C₁₋₄ alkyl. Preferably, R³ is selected from the group consisting of H and Me.

R⁴ is H, or an optionally substituted C₁₋₄ alkyl. Preferably, R⁴ is a C₁₋₄ alkyl optionally substituted with —OH. More preferably, R⁴ is selected from the group consisting of H, Me and CH₂OH.

R⁵ is H, or an optionally substituted C₁₋₄ alkyl. Preferably, R⁵ is selected from the group consisting of H and Me.

In some embodiments, R³ is selected from the group consisting of H and Me, R⁴ is selected from the group consisting of H, Me and CH₂OH, and R⁵ is selected from the group consisting of H and Me

Typical optional substituents include halogen, hydroxyl, nitro, carbonate, alkoxy, aryloxy, heteroaryloxy, amino, alkylamino, imine, nitrile, acetylide, or optionally substituted aliphatic, heteroaliphatic, alicyclic, heteroalicyclic, aryl or heteroaryl groups (for example, optionally substituted by halogen, hydroxyl, nitro, carbonate, alkoxy, amino, alkylamino, imine, nitrile or acetylide).

For the purpose of the present invention, an alkyl group is preferably a “C₁₋₆ alkyl group”, that is an alkyl group that is a straight or branched chain with 1 to 6 carbons. The alkyl group therefore has 1, 2, 3, 4, 5 or 6 carbon atoms. More preferably, an alkyl group is a “C₁₋₄ alkyl group”, that is an alkyl group that is a straight or branched chain with 1 to 4 carbons. The alkyl group therefore has 1, 2, 3 or 4 carbon atoms. Specifically, examples of “C₁₋₆ alkyl group” include methyl group, ethyl group, n-propyl group, iso-propyl group, n-butyl group, iso-butyl group, sec-butyl group, tert-butyl group, n-pentyl group, 1,1-dimethylpropyl group, 1,2-dimethylpropyl group, 2,2-dimethylpropyl group, 1-ethylpropyl group, n-hexyl group, 1-ethyl-2-methylpropyl group, 1,1,2-trimethylpropyl group, 1-ethylbutyl group, 1-methylbutyl group, 2-methylbutyl group, 1,1-dimethylbutyl group, 1,2-dimethylbutyl group, 2,2-dimethylbutyl group, 1,3-dimethylbutyl group, 2,3-dimethylbutyl group, 2-ethylbutyl group, 2-methylpentyl group, 3-methylpentyl group and the like.

A cycloalkyl group is preferably a “C₃₋₈ cycloalkyl group” that is a cycloalkyl group with 3 to 8 carbon atoms. The cycloalkyl group therefore has 3, 4, 5, 6, 7 or 8 carbon atoms. More preferably, a cycloalkyl group is a “C3-6 cycloalkyl group” that is a cycloalkyl group with 3 to 6 carbon atoms. The cycloalkyl group therefore has 2, 3, 4 or 6 carbon atoms. Specifically, examples of the C₃₋₈ cycloalkyl group include cyclopropyl, cyclobutyl, cyclopentyl, cyclohexyl, cycloheptyl and cyclooctyl. It will be appreciated that the cycloalkyl group may comprise a cycloalkyl ring bearing one or more linking or non-linking alkyl substituents, such as —CH₂-cyclohexyl.

The term “halide” used herein means a fluorine atom, a chlorine atom, a bromine atom, an iodine atom and the like, preferably a fluorine atom or a chlorine atom, and more preferably a fluorine atom.

An alkoxy group is preferably a “C₁₋₆ alkoxy group” and is an oxy group that is bonded to the previously defined “C₁₋₆ alkyl group”. Specifically, examples of “C₁₋₆ alkoxy group” include methoxy group, ethoxy group, n-propoxy group, iso-propoxy group, n-butoxy group, iso-butoxy group, sec-butoxy group, tert-butoxy group, n-pentyloxy group, iso-pentyloxy group, sec-pentyloxy group, n-hexyloxy group, iso-hexyloxy group, 1,1-dimethylpropoxy group, 1,2-1 dimethylpropoxy group, 2,2-dimethylpropoxy group, 2-methylbutoxy group, 1-ethyl-2-methylpropoxy group, 1,1,2-trimethylpropoxy group, 1,1-dimethylbutoxy group, 1,2-dimethylbutoxy group, 2,2-dimethylbutoxy group, 2,3-dimethylbutoxy group, 1,3-dimethylbutoxy group, 2-ethylbutoxy group, 2-methylpentyloxy group, 3-methylpentyloxy group and the like.

An aryl group is preferably a “C₆₋₁₂ aryl group” and is an aryl group constituted by 6, 7, 8, 9, 10, 11 or 12 carbon atoms and includes condensed ring groups such as monocyclic ring group, or bicyclic ring group and the like. Specifically, examples of “C₆₋₁₀ aryl group” include phenyl group, biphenyl group, indenyl group, naphthyl group or azulenyl group and the like. It should be noted that condensed rings such as indan and tetrahydro naphthalene are also included in the aryl group.

An alkylaryl group is preferably a “C₁₋₁₆ alkyl C₈₋₁₂ aryl group” and is an aryl group as defined above bonded at any position to an alkyl group as defined above. Preferably, the alkylaryl group is —CH₂-Ph or —CH₂CH₂-Ph.

A nitrile group is preferably a group CN or a group CNR⁷ wherein R⁷ is an alkyl group or an aryl group as defined above. Preferably R⁷ is an alkyl group selected from methyl, ethyl or propyl.

An alkenyl group contains a double bond —C═C—R⁹ wherein R⁹ can be an alkyl group or an aryl group as defined above. For the purposes of the invention when R⁹ is alkyl, the double bond can be present at any position along the alkyl chain. Preferably R⁹ is methyl, ethyl, propyl or phenyl.

An amino group is preferably NH₂, NHR⁹ or N(R⁹)₂ wherein R⁹ can be an alkyl group, a silylalkyl group or an aryl group as defined above. It will be appreciated that when the amino group is N(R⁹)₂, each R⁹ group can be independently selected from an alkyl group, a silylalkyl group or an aryl group as defined above. Preferably R⁹ is methyl, ethyl, propyl, SiMe₃ or phenyl.

An amido group is —NR¹⁰C(O)— or —C(O)—NR¹⁰— wherein R¹⁰ can be hydrogen, an alkyl group or an aryl group as defined above. Preferably R R¹⁰ is hydrogen, methyl, ethyl, propyl or phenyl.

A heterocycloalkyl group is a cycloalkyl group as defined above which has, in addition to carbon atoms, one or more ring heteroatoms, which are preferably selected from O, S, N, P and Si. Heterocycloalkyl groups preferably contain from one to four heteroatoms, which may be the same or different. Heterocycloalkyl groups preferably contain from 5 to 20 atoms, more preferably from 5 to 14 atoms, even more preferably from 5 to 12 atoms.

A heteroaryl group is an aryl group having, in addition to carbon atoms, from one to four ring heteroatoms which are preferably selected from O, S, N, P and Si. A heteroaryl group preferably has from 5 to 20, more preferably from 5 to 14 ring atoms. Specifically, examples of a heteroaryl group includes pyridine, imidazole, N-methylimidazole and 4-dimethylaminopyridine.

Examples of cycloalkyl, heterocycloalkyl, aryl and heteroaryl groups include but are not limited to cyclohexyl, phenyl, acridine, benzimidazole, benzofuran, benzothiophene, benzoxazole, benzothiazole, carbazole, cinnoline, dioxin, dioxane, dioxolane, dithiane, dithiazine, dithiazole, dithiolane, furan, imidazole, imidazoline, imidazolidine, indole, indoline, indolizine, indazole, isoindole, isoquinoline, isoxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, pyran, pyrazine, pyrazole, pyrazoline, pyrazolidine, pyridazine, pyridine, pyrimidine, pyrrole, pyrrolidine, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, and trithiane.

“Halo” as used herein includes fluoro, chloro, bromo and iodo. “Hetero” atoms may be selected from the group consisting of nitrogen, oxygen, sulphur, phosphorus, boron, chlorine, bromine and iodine. Suitably, the heteroatom is selected from the group consisting of nitrogen, oxygen and sulphur.

In some embodiments of the invention, the leukotriene antagonists have a structure according to any one of Formulae I to III. For example, the present invention provides a leukotriene antagonist for use as an antibiotic, wherein the leukotriene antagonist is a compound having the formula or any one of Formulae I to III. In particular, the leukotriene antagonist may have a formula according to Formula III.

In some embodiments of the invention, the leukotriene antagonist or compound of any one of Formulae I to III is not montelukast.

Suitably, the compounds of any one of Formulae I to III are leukotriene receptor antagonists, or prodrugs thereof. References to compounds of any one of Formulae I to III and to leukotriene receptor antagonists include pharmaceutically acceptable salts, esters, amides and prodrugs thereof.

Zafirlukast has the following structure:

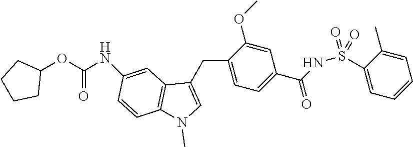 [SMILES: COc1cc(C(=O)NS(=O)(=O)c2ccccc2C)ccc1Cc1cn(C)c2ccc(NC(=O)OC3CCCC3)cc12]

The terms “leukotriene antagonists” and “leukotriene receptor antagonists” are used interchangeably. They refer to compounds that block the action of cysteinyl leukotrienes at the CysLT1 receptor on target cells such as bronchial smooth muscle. Such compounds are also known as “leukasts”.

In some embodiments of the invention, the compound used in the invention is zafirlukast or one of its metabolites, or a prodrug of zafirlukast. The systematic name for zafirlukast is [3-[2-Methoxy-4-(toluene-2-sulfonylaminocarbonyl)benzyl]-I-methyl-IH-indol-5-yl]carbamic acid cyclopentyl ester. Metabolites of zafirlukast for use in the invention include N-[4-(5-Amino-1-methyl-1H-indol-3-ylmethyl)-3-methoxybenzoyl]-2-methyl-benzenesulfonamide, N-[3-[2-Methoxy-4-(toluene-2-sulfonylaminocarbonyl)benzyl]-1H-indol-5-yl]acetamide, N-[1-Hydroxymethyl-3-[2-methoxy-4-(toluene-2-sulfonylaminocarbonyl)-benzyl]-1H-indol-5-yl]acetamide, N-[3-[2-Methoxy-4-(toluene-2-sulfonylaminocarbonyl)benzyl]-1-methyl-1H-indol-5-yl]acetamide, [3-[2-Methoxy-4-(toluene-2-sulfonylaminocarbonyl)benzyl]-IH-indol-5-yl]carbamic acid cyclopentyl ester, [3-[2-Methoxy-4-(toluene-2-sulfonylaminocarbonyl)benzyl]-1-methyl-IH-indol-5-yl]carbamic acid hydroxycyclopentyl ester, [1-Hydroxymethyl-3-[2-methoxy-4-(toluene-2-sulfonylaminocarbonyl)-benzyl]-1H-indol-5-yl]carbamic acid cyclopentyl ester and [3-[2-Methoxy-4-(toluene-2-sulfonylaminocarbonyl)benzyl]-1H-indol-5-yl]carbamic acid hydroxycyclopentyl ester.

Generally, in embodiments of the invention, the compounds used are leukotriene receptor antagonists. Zafirlukast is an exemplary compound for use in the present invention. Other leukotriene antagonists that can be used include pranlukast, MK-886 and zileuton. The compounds used in the invention may be in the form of pharmaceutically acceptable salts.

Additionally, the compounds used in the invention may be formulated as pharmaceutical compositions comprising a compound used in the invention (or pharmaceutically acceptable salts, esters or derivatives thereof) and one or more pharmaceutically acceptable excipients.

In some embodiments of the invention, the infection treated using a leukotriene antagonist or compound of any of Formulae I to III may be a skin, respiratory tract, alimentary canal or systemic infection. They may be infections in mammals, for example humans.

The compounds used in the present invention can be obtained by any suitable means known to a person of skill in the art. Some compounds, such as zafirlukast, are available for purchase from, for example, Sigma Aldrich (UK).

In embodiments of the invention, the leukotriene antagonists or compounds of any of Formulae I to III, or salts, esters or derivatives thereof, may be administered in combination with one or more other pharmaceutically active agents. The compounds or leukotriene antagonists may be for simultaneous, separate or sequential administration with another pharmaceutically active agent. Generally, the additional pharmaceutically active agents will be present in a separate preparation, although they may be present in the same pharmaceutical composition.

The compounds or leukotriene antagonists used in the invention may be administered in combination with one or more anti-inflammatory agents. Such anti-inflammatory agents include glucocorticoids, such as prednisone, prednisolone, dexamethasone, methylprednisolone, budesonide, hydrocortisone, betamethasone, triamcinolone or fludrocortisone. A preferred glucocorticoid may be prednisolone. Generally, the compounds used in the present invention will not be administered with NSAIDs.

The compounds or leukotriene antagonists used in the invention may be for administration in combination with asthma therapy. Such asthma therapies include glucocorticoids, 11-adrenergic agonists (short-acting, such as salbutamol, or long-acting, such as salmeterol or formoterol) or anti-cholinergic medications. In some embodiments, the zafirlukast is not administered with any such asthma therapies. Indeed, in some embodiments, the leukotriene antagonist, or compound of any one of Formulae I to III, is the only pharmaceutically active agent administered to the patient to treat the bacterial infection.

The compounds or leukotriene antagonists used in the invention may be administered in combination with one or more antibacterial agents. Such antibacterial agents include aminoglycosides, cephalosporins, macrolides, penicillins, quinolones, sulfonamides or tetracyclines. Aminoglycosides include amikacin, gentamycin, kanamycin, neomycin and tobramycin. Macrolides include azithromycin, clarithromycin, erythromycin or telithromycin. Penicillins include amoxicillin, ampicillin, flucloxacillin, methicillin, nafcillin, oxacillin, penicillin G, penicillin V or piperacillin. Quinolones include ciprofloxacin or levofloxacin. Tetracyclines include doxycycline and tetracycline. The additional antibiotics may be effective against Gram-positive bacteria. The additional antibiotics may be an antibiotic to which the bacterial infection is resistant, for example β-lactam antibiotics such as methicillin.

In other embodiments, the leukotriene antagonists, or compound of any one of Formulae I to III, is the only pharmaceutically active agents administered to the patient to treat the bacterial infection. In some embodiments, the compound use in the invention is the only antibiotic or antibacterial agent administered to the patient, although other pharmaceutically active agents may be administered.

In a second aspect of the invention there is provided a pharmaceutical composition comprising a leukotriene antagonist and one or more pharmaceutically acceptable excipients for use in the treatment of bacterial infections. There is also provided a pharmaceutical composition comprising a compound of any one of Formulae I to III and one or more pharmaceutically acceptable excipients, for use in the treatment of bacterial infections.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions).

Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. The excipient can be lactose or microcrystalline cellulose.

For the preparation of solutions and syrups, excipients which may be used include, for example water, polyols and sugars. For the preparation of suspensions oils (e.g. vegetable oils) may be used to provide oil-in-water or water in oil suspensions.

The leukotriene antagonists, such as those of any of Formulae I to III, are particularly suited to oral and systemic administration (such as administration by injection, including intravenous and/or intra-arterial administration). Administration by inhalation is also a preferred route (for example when formulated with lactose).

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3 (6), page 318 (1986).

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are suitably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas. Pharmaceutical compositions adapted for nasal administration wherein the carrier is a solid include a coarse powder having a particle size for example in the range 20 to 500 microns. Suitable compositions wherein the carrier is a liquid, for administration as a nasal spray or as nasal drops, include aqueous or oil solutions of the active ingredient. Pharmaceutical compositions adapted for vaginal administration may be presented as pessaries, tampons, creams, gels, pastes, foams or spray formulations. Pharmaceutical compositions adapted for administration by inhalation include powders and fine particle dusts or mists which may be generated by means of various types of metered dose pressurised aerosols, nebulizers or insufflators.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention. References herein to leukotriene antagonists include pharmaceutically acceptable salts or esters thereof. Similarly, references to compounds of any of Formulae I to III include pharmaceutically acceptable salts or esters thereof. Such salts include hydrochloride (HCl), mesylate, maleate, chloride, bromide, citrate, tartrate, sulphate, and phosphate, including any suitable cation (sodium, calcium, benzathine, magnesium, ammonium, zinc, potassium and so on). Compounds used in the invention may include a carboxylic acid group; for such compounds, a salt can be formed by decomposition of the carboxylic acid to form a carbon/late.

In embodiments in which the compounds used in the invention are formulated as compositions for oral administration, the compositions may further comprise croscarmellose sodium, lactose, magnesium stearate, microcrystalline cellulose, povidone, hypromellose, and titanium dioxide. The compositions may be tablets, such as film-coated tablets. In embodiments of the invention in which the compounds used are formulated as compositions for oral administration, they may be formulated as sustained- or controlled-release formulations.

Dosages of the pharmaceutical compositions of the present invention can vary between wide limits, depending upon the disease or disorder to be treated, the age and condition of the individual to be treated, etc. and a physician will ultimately determine appropriate dosages to be used. Such compositions may be formulated for human or for veterinary medicine. The present application should be interpreted as applying equally to humans as well as to animals, unless the context clearly implies otherwise.

In embodiments in which the compounds used in the invention are formulated as compositions for oral administration, the compositions may comprise between 5 and 50 mg of the leukotriene receptor antagonists (or a compound of any one of Formulae I to III), for example between 10 and 20 mg. In some embodiments, the amount to be administered may be between 0.05 and 100 mg/kg, for example between 0.05 and 50 mg/kg, 0.05 and 25 mg/kg, 0.05 and 5 mg/kg, 0.05 and 1 mg/kg or 0.05 and 0.5 mg/kg.

The pharmaceutical compositions may further comprise additional pharmaceutically active agents, for example antibacterial agents, anti-inflammatory agents or agents used in asthma therapy. In other embodiments, the leukotriene antagonists, or compound of any one of Formulae I to III, is the only pharmaceutically active agents present in the pharmaceutical composition.

The invention extends to methods of manufacture of suitable pharmaceutical compositions, as well as the use of leukotriene antagonists (such as zafirlukast) or the compounds of any one of Formulae I to III in the manufacture of a medicament for the treatment of bacterial infection, or indeed for any of the uses specified herein.

In a third aspect of the invention, there is provided a method of treating or preventing a bacterial infection in a patient, comprising administering a leukotriene antagonist (such as zafirlukast), or a compound of any one of Formulae I to III (or a pharmaceutical composition comprising a leukotriene antagonist (such as zafirlukast) or a compound of any one of Formulae I to III) to a patient in need thereof. The compound or pharmaceutical composition may be administered by any suitable route, for example the oral route or systemic route (such as by injection, for example intravenous injection), or by inhalation. Additional pharmaceutically active agents may also be administered to treat the bacterial infection. In other embodiments, the leukotriene antagonist or a compound of any one of Formulae I to III may be the only pharmaceutically active agent administered to the patient to treat the bacterial infection.

Methods of treatment may include a step of selecting a patient for treatment. For example, the method may include a step of determining whether a patient will be susceptible to treatment. Such steps may include first determining the presence (or absence) of a bacterial infection and administering the compound used in the invention only if a bacterial infection is detected. In particular, treatment might only be initiated if the patient is infected with a certain type of bacterial infection, for example infection by Gram-positive bacteria, a Firmicute or a Gram-positive Firmicute. Nosocomial infections, including nosocomial Gram-positive nosocomial infections, may be targeted in the present invention.

The decision whether or not to treat may be based on the present of an antibiotic-resistant bacterial infection, or even multidrug-resistant strains. Specific infections that can be treated include Staphylococcus aureus, methicillin resistant Staphylococcus aureus, Enterococcus faecalis, Bacillus subtilis, Streptococcus pneumoniae and Clostridium difficile infections. In some embodiments, treatment may be initiated if an infection with bacterial species that do not encode Lsr2, or an Lsr2-homologue, is detected. In some embodiments, treatment is not initiated if a Mycobacterium spp. or Corynebacterium spp. infection is detected or suspected. In other embodiments, treatment is not initiated if an actinobacterial infection is detected or suspected, for example a Streptomyces spp., Nocardia spp. or Rhodococcus spp. infection.

The step of determining the presence of a bacterial infection may include testing for the infection in question. It may include testing for the presence of an infection by Gram-positive bacteria, for example an infection by Staphylococcus aureus, methicillin resistant Staphylococcus aureus, Enterococcus faecalis, Bacillus subtilis, Streptococcus pneumoniae or Clostridium difficile, and administering a compound used in the invention accordingly if such an infection is detected or suspected. In other embodiments, the step of determining the presence of a bacterial infection may include testing for the presence of a Mycobacterium spp. and/or Corynebacterium spp. infection, with the decision to treat the patient by administering a compound used the invention only being taken in the absence of such infection. In other embodiments, the step of determining the presence of a bacterial infection may include testing for the presence of an actinobacterial, for example a Streptomyces spp., Nocardia spp. or Rhodococcus spp. infection, with the decision to treat the patient by administering a compound used the invention only being taken in the absence of such infection.

In a fourth aspect of the invention there is provided the use of a leukotriene antagonist, or the use of a compound of any one of Formulae I to III, or the use of a pharmaceutical composition comprising a leukotriene antagonist or a compound of any one of Formulae I to III, in the manufacture of a medicament for the treatment of bacterial infection.

In a further aspect of the invention there is provided a leukotriene antagonist, or a compound of any one of Formulae I to III, or a pharmaceutical composition comprising a leukotriene antagonist or a compound of any one of Formulae I to III, for use in the treatment of a bacterial infection accompanying inflammatory lung disease or (chronic) asthma, or obstructive lung diseases. The compound used in the invention is administered as an antibiotic, in particular a bactericidal antibiotic. The inflammatory lung disease may be asthma, cystic fibrosis, emphysema, chronic obstructive pulmonary disorder (COPD) or acute respiratory distress syndrome (ARDS). In some embodiments, the respiratory tract infection is an upper respiratory tract infection. In other embodiments, the respiratory tract infection is a lower respiratory tract infection. In some embodiments, the disease is not tuberculosis. Generally, the bacterial infection accompanying inflammatory lung disease or chronic asthma, or obstructive lung diseases is not a Mycobacterium or Corynebacterium infection.

In another aspect of the invention, there is provided a method of inhibiting the growth of bacteria comprising the administration of a leukotriene antagonist or compound of any one of Formula I to III. There is also provided a method of lysing bacteria comprising administering a leukotriene antagonist or compound of any one of Formula I to III. Such methods may be in vivo or in vitro. Generally, the bacteria whose growth is inhibited or are lysed are not Mycobacterium spp. or Corynebacterium spp., and in some embodiments they are not actinobacteria (for example a Streptomyces spp., Nocardia spp. or Rhodococcus spp.).

In some embodiments the leukotriene antagonist or compound of any one of Formula I to III is bactericidal. Methods of killing bacteria are therefore also including in this invention, as are the uses of leukotriene antagonists and the compounds of any one of Formula I to in such methods. For example, the method of killing bacteria may include contacting the bacteria (such as a Firmicute) with a compound or pharmaceutical composition of the invention (a leukotriene receptor antagonist or a compound having a formula according to any one of formulae I to III, such a zafirlukast). The bacteria may be infection a mammal, for example a human. Alternatively, the method may be a method of sterilisation or decontamination, in which case there might not be any pathological infection being treated.

The compounds, leukotriene antagonists or pharmaceutical compositions may be used in methods of preventing, reducing, inhibiting and/or controlling bacterial infections, as well as treating such infections. They may be for prophylactic administration.

There is also provided a kit of parts comprising a leukotriene antagonist or compound of any one of Formula I to III and an additional pharmaceutically active agent for use in the treatment of a bacterial infection. In some embodiments, the additional pharmaceutically active agent is not an antibiotic. The kit may further comprise instructions for use.

In one embodiment of the invention there is provided zafirlukast for use in in the treatment of a bacterial infection, wherein the bacteria is selected from the group consisting of Staphylococcus aureus, methicillin resistant Staphylococcus aureus, Enterococcus faecalis, Bacillus subtilis, Streptococcus pneumoniae, and Clostridium difficile. The zafirlukast is for oral administration to a patient in need thereof.

Preferred features for the second and subsequent aspects of the invention are as provided for the first aspect, mutatis mutandis.

The present invention will now be described by way of reference to the following Examples which are present for the purposes of reference only and are not to be construed as being limiting on the invention. In the examples, reference is made to a number of drawings in which:

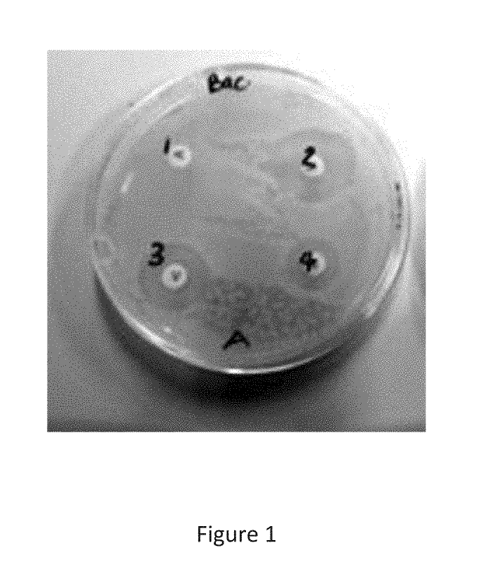

FIG. 1 shows the effect of decreasing amounts of zafirlukast on Bacillus subtilis. In the figure, 1=DMSO alone (control) and 2 to 4=decreasing amounts of zafirlukast (10, 5 and 2 μg, respectively).

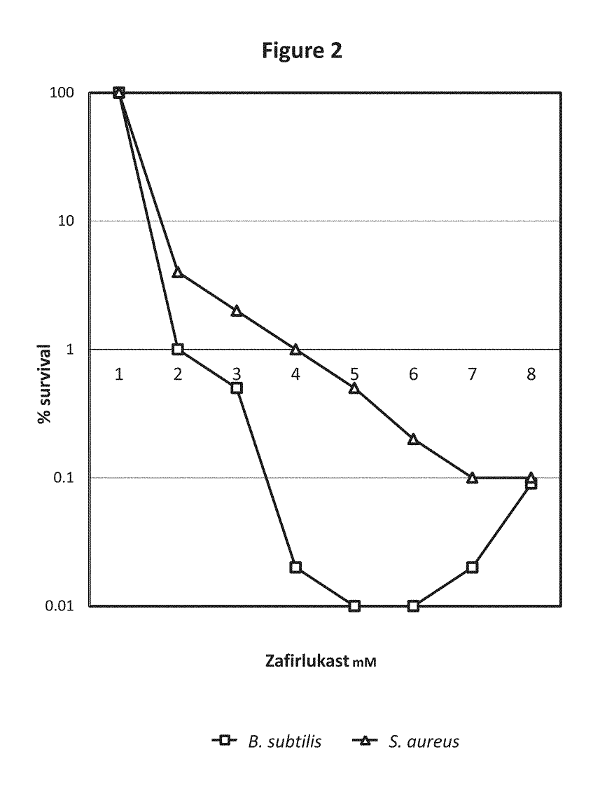

FIG. 2 shows the bactericidal activity of zafirlukast on S. aureus and B. subtilis.

### EXAMPLE 1

Zafirlukast (Sigma Aldrich, UK) was resuspended in Dimethyl Sulfoxide to a concentration of 16 mM. A minimum inhibitory concentration (MIC) was conducted using various bacterial broth solutions containing between the 0 and 4 mM zafirlukast. 10⁵ cfu/mL of test organism was added to each tube before incubation overnight at 37° C. The MIC was taken to be the concentration at which no visible growth was observed. In addition, zone inhibition studies were carried out using standard techniques. The results are shown in FIG. 1.

The bacterial species tested included Staphylococcus aureus, methicillin resistant Staphylococcus aureus, Enterococcus faecalis, Bacillus subtilis, Streptococcus pneumoniae, and Clostridium difficile.

The MICs were as follows:

Staphylococcus aureus:

Zafirlukast=0.03 mM

Ciprofloxacin=0.003 mM

Ceftriaxone=0.002 mM

Bacillus subtilis

Zafirlukast=0.006 mM

Ceftriaxone=>0.029 mM

Ciprofloxacin=>0.012 mM,

MRSA

Zafirlukast=0.006 mM

Ciprofloxacin=average 0.04 mM

Ceftriaxone=0.022 mM

MIC is well known to the skilled person and is defined as the lowest concentration of agent which prevents visible growth of the bacterial strain under investigation.

### EXAMPLE 2

Bactericidal Activity of Zafirlukast.

105 cfu/mL of bacterial culture was incubated in the presence of various concentrations of zafirlukast for a period of 3 hrs at 37° C. At the end of this incubation, viable counts were taken and results recoded as percentage survival (as compared to the drug free control). The results are shown in FIG. 2.

1. A method of treating a Gram-positive bacterial infection in a patient in need thereof, comprising administering a therapeutically effective amount of a leukotriene receptor antagonist, or a pharmaceutically acceptable salt or ester thereof, wherein the bacterial infection is not a Mycobacterium or Corynebacterium infection.

2. The method of claim 1, wherein the leukotriene receptor antagonist is selected from the group consisting of zafirlukast, pranlukast, MK-886 and zileuton, and derivatives or prodrugs thereof.

3. A method of treating a Gram-positive bacterial infection in a patient in need thereof, comprising administering a therapeutically effective amount of a compound of Formula I 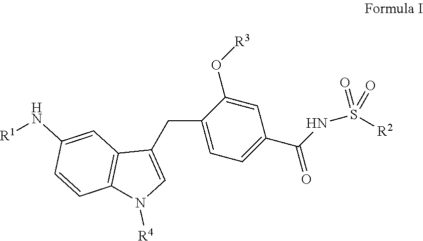
wherein:
R¹ is H, an optionally substituted C₁₋₄ alkyl, C(O)R′ or C(O)OR″;
R′ is H or an optionally substituted C₁₋₄ alkyl or —NR^{III}R^{IV};
wherein R^{III} and R^{IV} are independently H, an optionally substituted C₁₋₄ alkyl, or are taken together to form a heterocyclic ring;
R″ is H, an optionally substituted C₁₋₆ alkyl or an optionally substituted C₃₋₆ cycloalkyl or heterocycloalkyl;
R² is H, or an optionally substituted alkyl, cycloalkyl or heterocycloalkyl, aryl or heteroaryl;
R³ is H, or an optionally substituted C₁₋₄ alkyl; and
R⁴ is H, or an optionally substituted C₁₋₄ alkyl,
or a pharmaceutically acceptable salt, ester, amide or prodrug thereof,
wherein the bacterial infection is not a Mycobacterium or Corynebacterium infection.

4. The method of claim 3, wherein R¹ is selected from the group consisting of H, C(O)Me and C(O)OR″, wherein R″ is H or a cycloalkyl optionally substituted with OH and R² is an aryl optionally substituted with CH₃.

5. The method of claim 3, wherein R¹ is selected from the group consisting of H, C(O)Me and C(O)OR″, wherein R″ is H or a cycloalkyl (preferably C₅) optionally substituted with OH, R² is an aryl optionally substituted with CH₃, R³ is selected from the group consisting of H and Me and R⁴ is selected from the group consisting of H, Me and CH₂OH.

6. The method of claim 3, wherein the compound is a compound of Formula II 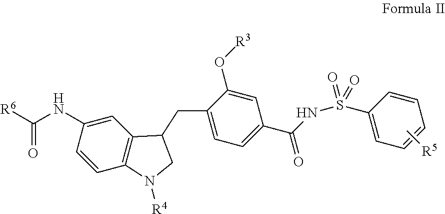
wherein:
R³ is H, or an optionally substituted C₁₋₄ alkyl;
R⁴ is H, or an optionally substituted C₁₋₄ alkyl;
R⁵ is H, or an optionally substituted C₁₋₄ alkyl; and
R⁶ is H, a C₁₋₄ alkyl, a heterocycloalkyl or OR′″, wherein R′″ is H, C₁₋₆ alkyl or an optionally substituted C₃₋₆ cycloalkyl,
or a pharmaceutically acceptable salt, ester, amide or prodrug thereof.

7. The method of claim 6, wherein R³ is selected from the group consisting of H and Me, R⁴ is selected from the group consisting of H, Me and CH₂OH, R⁵ is H or Me and R⁶ is OR′″, wherein R′″ is a C₃₋₆ cycloalkyl optionally substituted with OH.

8. The method of claim 3, wherein the compound is a compound of Formula III 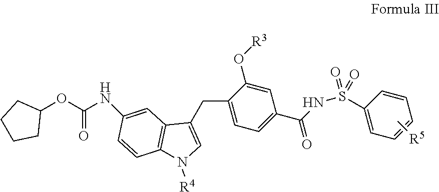
wherein:
R³ is H, or an optionally substituted C₁₋₄ alkyl;
R⁴ is H, or an optionally substituted C₁₋₄ alkyl; and
R⁵ is H, or an optionally substituted C₁₋₄ alkyl,
or a pharmaceutically acceptable salt, ester, amide or prodrug thereof.

9. The method of claim 8, wherein R³ is selected from the group consisting of H and Me, R⁴ is selected from the group consisting of H, Me and CH₂O, and R⁵ is selected from the group consisting of H and Me.

10. The method of claim 1, wherein the compound is zafirlukast or a prodrug, metabolite or derivative thereof.

11. The method of claim 10, wherein the metabolite or derivative of zafirlukast is N-[4-(5-Amino-1-methyl-1H-indol-3-ylmethyl)-3-methoxybenzoyl]-2-methyl-benzenesulfonamide, N-[3-[2-Methoxy-4-(toluene-2-sulfonylaminocarbonyl)benzyl]-1H-indol-5-yl]acetamide, N-[1-Hydroxymethyl-3-[2-methoxy-4-(toluene-2-sulfonylaminocarbonyl)-benzyl]-1H-indol-5-yl]acetamide, N-[3-[2-Methoxy-4-(toluene-2-sulfonylaminocarbonyl)benzyl]-1-methyl-1H-indol-5-yl]acetamide, [3-[2-Methoxy-4-(toluene-2-sulfonylaminocarbonyl)benzyl]-1H-indol-5-yl]carbamic acid cyclopentyl ester, [3-[2-Methoxy-4-(toluene-2-sulfonylaminocarbonyl)benzyl]-1-methyl-1H-indol-5-yl]carbamic acid hydroxycyclopentyl ester, [1-Hydroxymethyl-3-[2-methoxy-4-(toluene-2-sulfonylaminocarbonyl)-benzyl]-1H-indol-5-yl]carbamic acid cyclopentyl ester and [3-[2-Methoxy-4-(toluene-2-sulfonylaminocarbonyl)benzyl]-1H-indol-5-yl]carbamic acid hydroxycyclopentyl ester.

12. The method of claim 1 or claim 3, wherein the bacterial infection is an opportunistic or nosocomial bacterial infection.

13. The method of claim 1 or claim 3, wherein the bacterial infection is infection with a bacterial species that does not encode Lsr2 or a homologue thereof.

14. The method of claim 1 or claim 3, wherein the infection is selected from the group consisting of a Staphylococcus spp., an Enterococcus spp., a Bacillus spp., a Streptococcus spp. and a Clostridium spp. infection.

15. The method of claim 1 or claim 3, wherein the infection is selected from the group consisting of a Staphylococcus aureus, methicillin resistant Staphylococcus aureus, Enterococcus faecalis, Bacillus subtilis, Streptococcus pneumoniae, and Clostridium difficile infection.

16. The method of claim 1 or claim 3, wherein the leukotriene receptor antagonist or compound is administered orally or systemically, or by inhalation.

17. The method of claim 1 or claim 3, wherein the leukotriene receptor antagonist or compound is administered simultaneously, separately or sequentially with another pharmaceutically active agent.

18. The method of in claim 17, wherein the pharmaceutically active agent is an antibacterial agent, an anti-inflammatory agent, or an agent used in asthma therapy.

19. The method of claim 1, wherein the leukotriene antagonist is administered in the form of a pharmaceutical composition comprising the leukotriene antagonist and one or more pharmaceutically acceptable excipients.

20. The method of claim 3, claim 6 or claim 8, wherein the compound of Formula I, II or III is administered in the form of a pharmaceutical composition comprising the compound of any one of Formula I, II or III, and one or more pharmaceutically acceptable excipients.

21-36. (canceled)


SEQUENCE LISTING (1 sequence)

SEQ ID NO: 1 - protein, 112 aa, Mycobacterium tuberculosis
MAKKVTVTLVDDFDGSGAADETVEFGLDGVTYEIDLSTKNATKLRGDLKQWVAAGRRVGGRRRGRSGSGRGRGAIDREQSAAIREWARRNGHNVSTRGRIPADVIDAYHAAT